(19) United States
(12) Patent Application Publication
(10) Pub. No.: US 2010/0208910 A1
(43) Pub. Date: Aug. 19, 2010

(54) ACOUSTIC FIELD CORRECTION METHOD AND AN ACOUSTIC FIELD CORRECTION DEVICE

(75) Inventor: Yasunori Ohora, Yokohama-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/703,715

(22) Filed: Feb. 10, 2010

(30) Foreign Application Priority Data
Feb. 18, 2009 (JP) 2009-035814

(51) Int. Cl. A61F 11/06

(52) U.S. Cl. 381/71.8

(57) ABSTRACT

An acoustic field correction device corrects effect of frequency characteristics by indoor standing waves, determines a frequency range in which resonance due to standing waves occur, and adjusts an attenuation amount of a filter which suppresses the determined frequency range. In this adjustment, adjustment is made to reduce the amount of attenuation at the time of initial rise of the signal in the frequency range of the standing wave.

### BACKGROUND OF THE INVENTION

1. Field of the Invention

The present invention relates to a technique of acoustic field correction for correcting influence of frequency characteristics by indoor standing waves.

2. Description of the Related Art

When generating sound from a sound source such as a speaker in an indoor setting such as a home, reflected sound from various surfaces such as walls, roof and floor of a room are generated in addition to the direct sound which arrives to various places in the room via the shortest distance, resulting in overlap of sounds. In such a situation, when the distance between two parallel surfaces facing each other is an integral multiple of the half-wavelength of the sound wave, a standing wave is generated which leads to resonance in a low-frequency range referred to as “booming”.

In such a situation, suppression of booming with a parametric equalizer, and correction using waves having properties which are opposite to the standing wave by measuring its acoustic properties with a microphone at a listening position, are performed. In addition to these techniques, a method of using directional information of reflected sound is also described (for example, Japanese Patent Laid-Open No. 05-83786).

Generally, standing waves are generated by overlapping of reflected sound. For example, when a sound wave having a wave profile shown in FIG. 1 is output from a speaker, amplitude of the standing wave grows with time in a room. As a result, the sound wave at the listening point will have a wave profile which is shown in FIG. 2. However, with conventional correction methods, problems such as those shown below exist.

When performing correction with a conventional parametric equalizer, the amount of attenuation at the wavelength is fixed regardless of time. For this reason, booming can be suppressed, but it takes time for reducing the sound to a certain volume. It also gives an impression that the sound of the wavelength is generated later when compared to sounds of other wavelengths.

Further, even when acoustic properties are measured with a microphone at the listening position and correction is done using opposite properties, the properties of correction do not change with time, which leads to an impression that sound of the wavelength is generated later than sounds of other wavelengths.

### SUMMARY OF THE INVENTION

The present invention provides a method and a device which enable correction of the initial rise of a signal in a frequency range of the standing wave.

According to one aspect of the present invention, there is provided an acoustic field correction method which corrects effects of frequency characteristics by indoor standing waves, comprising: determining a frequency range in which resonance by the standing waves are generated; and adjusting an amount of attenuation of a filter which suppresses the frequency range determined in the determination step, wherein the adjustment is made in the adjustment step to reduce the amount of attenuation at the time of initial rise of the signal in the frequency range of the standing wave.

According to another aspect of the present invention, there is provided an acoustic field correction device which corrects effect of frequency characteristics by indoor standing waves, comprising: a determination unit that determines a frequency range in which resonance by the standing waves are generated; and an adjustment unit that adjusts an amount of attenuation of a filter which suppresses the frequency range determined by the determination means, wherein the adjustment is made by the adjustment unit to reduce the amount of attenuation at the time of initial rise of the signal in the frequency range of the standing wave.

Further features of the present invention will become apparent from the following description of exemplary embodiments (with reference to the attached drawings).

### BRIEF DESCRIPTION OF THE DRAWINGS

FIG. 1 shows an example of a wave profile inputted to a speaker.

FIG. 2 shows a sound pressure wave profile at a listening point.

FIG. 3 is a block diagram showing an exemplary configuration of an acoustic field correction device according to the present embodiment.

FIG. 4 is a diagram which explains internal operations at wave profile envelope temporal differentiation calculating units and at peak hold units.

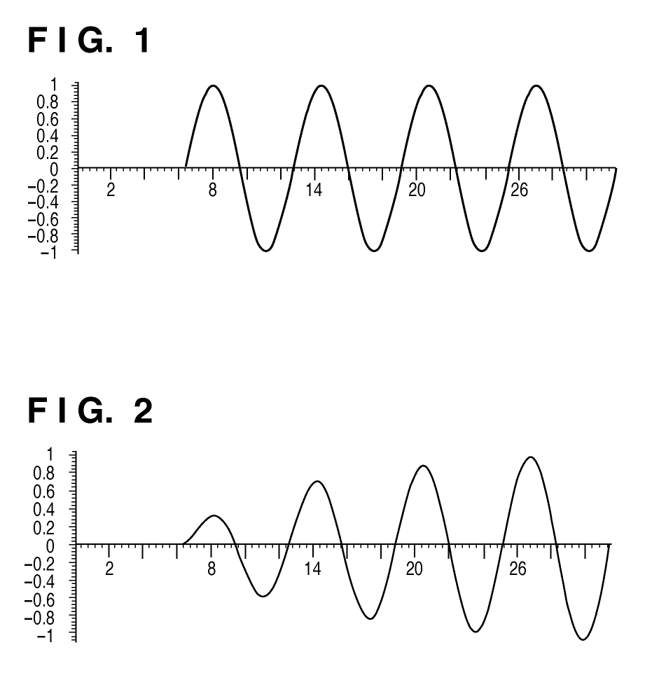

### DESCRIPTION OF THE EMBODIMENTS

Below, the most preferred embodiments of the present invention will be explained in detail.

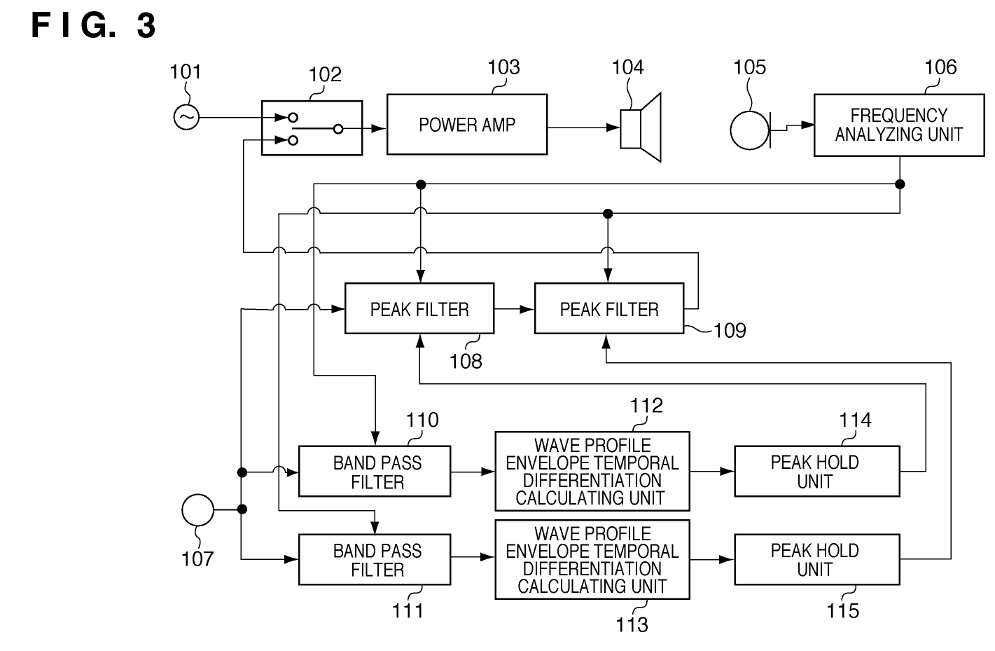

Firstly, the structure of an acoustic field correction device which performs an acoustic correction method according to the present invention will be explained with reference to FIG. 3.

FIG. 3 is a block diagram showing an exemplary configuration of an acoustic field correction device according to the present embodiment. An oscillator 101 shown in FIG. 3 generates white noise and sweep signal. An input selection switch 102 is a switch which selects one of two input signals. A power amp 103 amplifies the input signals such that they can be driven at a speaker 104. The speaker 104 is used to play back input signals as sound. A microphone 105 is used for monitoring the acoustics of the sound generated by the speaker 104. A frequency analyzing unit 106 performs frequency analysis of sound signal obtained from the microphone 105.

A signal input port 107 is a port for inputting music signal. Peak filters 108 and 109 are filters which suppress only a certain and very narrow range of frequency. Band pass filters 110 and 111 are filters which pass through a certain range of frequency. Wave profile envelope temporal differentiation calculating units 112 and 113 calculate wave profile envelope of the outputs from the band pass filters 110 and 111, and then calculates the initial rise of the signal by performing temporal differentiation. Peak hold units 114 and 115 add attenuation to the initial rise wave signals calculated by the wave profile envelope temporal differentiation calculating units 112 and 113.

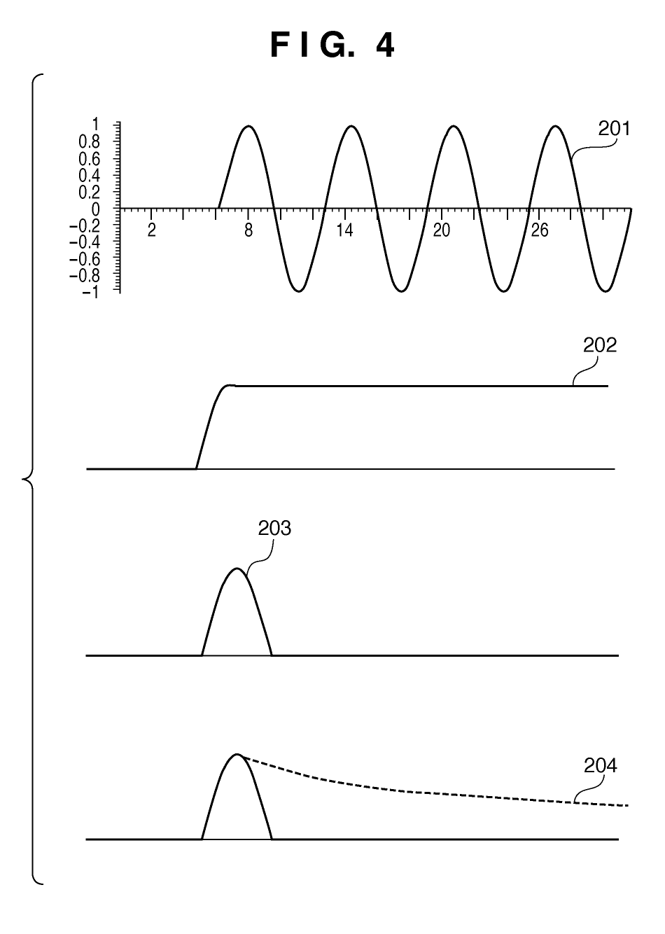

Next, explanation regarding operation that takes place within the wave profile envelope temporal calculating units 112 and 113 and peak hold units 114 and 115, shown in FIG. 3, will be provided with reference to FIG. 4.

FIG. 4 is a diagram which explains internal operations at the wave profile envelope temporal differentiation calculating units and at peak hold units. Here, numeral 201 refers to the wave profile inputted to the wave profile envelop temporal differentiation calculating unit 112 (113). Numeral 202 refers to an envelope of the wave profile 201 calculated by the wave profile envelope temporal differentiation calculating unit 112 (113). Numeral 203 refers to a temporally differentiated wave profile of the envelope 202 calculated at the wave profile envelope temporal differentiation calculating unit 112 (113). Numeral 204 is a wave profile hold-processed to a temporally differentiated wave profile 203 at the peak hold unit 114 (115).

Next, the flow of protocol 1, in which the conditions within the room are measured and a first and a second frequencies generated by the resonance of standing waves based on the measurement result are determined, will be explained in detail. In the present embodiment, by performing this protocol 1 and protocol 2 to be explained later, it is possible to obtain sound with good initial rising characteristics even at booming frequencies.

Firstly, prior to measuring conditions of the standing wave in the room, the input selection switch 102 is set to the side of the oscillator 101. Then, when the oscillator 101 is started, the white noise and sweep signals, which cover the frequency range that can be generated by the speaker 104, are generated and sent to the power amp 103. The power amp 103 performs signal amplification which is enough for generating adequate sound volume in the room, and drives the speaker 104.

The sound emanated from the speaker 104 arrives at the microphone 105 while being affected by reflections within the room. Frequency characteristics of the signals obtained at the microphone 105 are analyzed at the frequency analyzing unit 106. In order to simplify the explanation, only 2 standing waves are shown in FIG. 3. However, there can be more than this. The 2 frequency characteristics of the standing waves determined by the frequency analyzing unit 106 are designated as standing wave 1 and standing wave 2.

At this point, the information regarding the standing wave 1 is sent to the peak filter 108, and preparation is made to suppress the determined first frequency range. At the same time, the information is also sent to the band pass filter 110, and preparation is made to retrieve only the determined first frequency range.

At the same time, the information regarding the standing wave 2 is sent to the peak filter 109, and preparation is made to suppress the determined second frequency range. Simultaneously, the information is also sent to the band pass filter 111, and preparation is made to retrieve only the determined second frequency range.

Next, the flow of protocol 2, in which music is played back using the determined first and second frequencies, will be explained.

In order to actually play back music signals, the input selection switch 102 is set to the side of the peak filter 109. Then, a device such as a CD player is connected to the signal input port 107, and music signal is inputted into the signal input port 107. This signal is simultaneously sent to the peak filter 108, the band pass filter 110 and the band pass filter 111. The band pass filter 110 retrieves the signal of the first frequency range determined by the standing wave 1 of the music signal, and sends it to the wave profile envelope temporal differentiation calculating unit 112.

The wave profile 201 inputted into the envelope temporal differentiation calculating unit 112 obtained by retrieval of a specific frequency, has a wave profile which is close to that of a sinusoidal wave. When an envelope 202 is calculated from the wave profile 201, there are several methods of calculating the envelope that can be employed, and the Hilbert transform is commonly used. Of course, a wave detection method wherein the absolute value is obtained and passed through the low pass filter can also be used.

Next, after the envelope of the wave profile 201 is differentiated, the wave profile envelope temporal differentiation calculating unit 112 removes the negative portions from the result and sends it to the peak hold unit 114 as a temporally differentiated wave profile 203. This temporally differentiated wave profile 203 becomes the signal which indicates the initial rise of the signal in the first frequency range.

On the other hand, the peak hold unit 114 generates a wave profile shown by a dashed line 204 using hold processing having attenuation characteristics. The wave profile which is hold-processed (indicated by the dashed line 204) has opposing properties which cancel out the effects of the standing waves shown in FIG. 2. Further, a process which is identical to the above mentioned process is repeated for the range of the second wavelength using the band pass filter 111, the wave profile envelope temporal differentiation calculating unit 113, and the peak hold unit 115.

Then, the wave profile obtained from the peak hold units 114 and 115 is sent to the peak filters 108 and 109 as a gain adjustment curve. At the peak filter 108, gain adjustment of the first frequency range component from the inputted music signal is performed according to the instruction (gain adjustment curve) of the peak hold unit 114. At the same time, at the peak filter 109, gain adjustment of the second frequency range component from the inputted music signal filter processed at the peak filter 108 is performed according to the instruction (gain adjustment curve) of the peak hold unit 115.

In this manner, adjustment is made in advance to reduce the amount of attenuation of the peak filters at the time of initial rise of the signals in the frequency range of standing waves which will be affected by the emissions of the speaker 104. As a result, the characteristics of the initial rise in the frequency range of the standing waves at the listening point are improved.

As explained above, according to the present invention, even in the booming frequency, it is possible to attain sound having good initial rise characteristics.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-035814, filed Feb. 18, 2009 which is hereby incorporated by reference herein in its entirety.

What is claimed is:

1. An acoustic field correction method which corrects effects of frequency characteristics by indoor standing waves, comprising:
determining a frequency range in which resonance by the standing waves are generated; and
adjusting an amount of attenuation of a filter which suppresses the frequency range determined in the determination step,
wherein the adjustment is made in the adjustment step to reduce the amount of attenuation at the time of initial rise of the signal in the frequency range of the standing wave.

2. The method according to claim 1, wherein in the determining step, a frequency generated by a speaker is analyzed, and a frequency range which is generated by resonance of the standing waves is determined.

3. The method according to claim 1, wherein in the adjusting step, an envelope from a wave profile of a signal in the determined frequency range is calculated, and the amount of attenuation of the filter is adjusted based on a wave profile that the envelope is differentiated by temporal differentiation.

4. The method according to claim 1, wherein the amount of attenuation of said filter is an amount of gain adjustment of the filter.

5. An acoustic field correction device which corrects effect of frequency characteristics by indoor standing waves, comprising:
a determination unit that determines a frequency range in which resonance by the standing waves are generated; and
an adjustment unit that adjusts an amount of attenuation of a filter which suppresses the frequency range determined by said determination means,
wherein the adjustment is made by the adjustment unit to reduce the amount of attenuation at the time of initial rise of the signal in the frequency range of the standing wave.

6. A computer readable storage medium which stores a program for causing a computer to execute each of the steps of the acoustic field correction method according to claim 1.
